(19) United States
(12) Patent Application Publication
(10) Pub. No.: US 2004/0062795 A1
(43) Pub. Date: Apr. 1, 2004

(54) Multiple medical patches on a single strip and a process for manufacturing thereof

(76) Inventor: Alan E. Nash, Canton, MA (US)

Correspondence Address: Steven N. Fox, Esq., P.O. Box 251, Canton, MA, 02021, US

(21) Appl. No.: 10/430,891

(22) Filed: May 6, 2003

Related U.S. Application Data
(63) Continuation-in-part of application No. 09/739,585, filed on Dec. 18, 2000, now Pat. No. 6,559,455.

(51) Int. Cl. A61L 15/16; A61K 9/70

(52) U.S. Cl. 424/449; 424/447

(57) ABSTRACT

An article of manufacture for use in removing corns and callus from a person comprising a first strip comprising a backing member and first and second medicated patches having salicylic acid releaseably affixed to the backing member. Each of the first and second mediated patches comprise first and second outside edges and an inside edge. The first and second medicated patches are nested and adjoined along the inside edges of said first and second medicated patches, respectively.

### RELATED PENDING APPLICATION

This application claims priority to application Ser. No. 09/739,585 filed on Dec. 18, 2000, now pending.

### FIELD OF THE INVENTION

The present invention relates generally to the field of mediated patches for use by a person, and in particular, persons who need relief from painful corns and callus.

### BACKGROUND OF THE INVENTION

Conventional medicated pads are difficult and costly to manufacture, including, a loss in a significant amount of waste during the manufacturing process.

### SUMMARY OF THE PRESENT INVENTION

One object of the present invention was to provide an article of manufacture having multiple medicated patches on a single strip which can be easily peeled off and attached to the skin of a person; and

Another object of the present invention was to provide a process for manufacturing an article of manufacture having for multiple medicated patches on a single strip which can be easily peeled off and attached to the skin of a person and can be manufactured in a cost effective manner with substantially zero or no waste.

In one embodiment, the present invention is an article of manufacture for use in removing corns and callus from the skin of a person. The article comprises a first strip comprising a backing member and first and second medicated patches made from a non-woven fabric and coated on one side with a blend of salicylic acid and adhesive and releaseably affixed to the backing member. Each of the first and second medicated patches comprise first and second outside edges, and an inside edge. The first and second medicated patches are nested and adjoined along the inside edges of the first and second medicated patches, respectively. The first and second edges are non-planar. The inside edge of the first medicated patch comprises first and second peaks and first and second valleys. The inside edge of the second medicated patch comprises first and second peaks and first and second valleys. The first and second peaks of the inside edge of the first medicated patch are nested and adjoined within the first and second valleys of the inside edge of the medicated second patch. The backing member comprises a release material coated thereon to allow the medicated patches to be easily peeled off and affixed to the skin.

### BRIEF DESCRIPTION OF THE DRAWINGS

The following detailed description of the invention will be better understood with reference to the accompanying drawings in which:

FIG. 1 is a perspective view of a first embodiment of the present invention showing a single strip having a plurality of UV sensitive patches releaseably attached to a backing member;

FIG. 2 is a top plan view showing a plurality of UV sensitive patches releaseably attached to a backing member;

FIG. 3 is a cross-section view of the single strip of the present invention;

FIG. 4 is a perspective view of a second embodiment of the present invention showing the single strip disposed within a sealed enclosure;

FIG. 5 is a perspective view of a continuous roll of material used in connection with the manufacture of the present invention;

FIGS. 6A-6C are illustrative view showing the rotary cutting die positions in the first complete cutting operation and the second complete cutting operation;

FIG. 7 is a illustrative view showing one pattern of the rotary cutting die used in connection with the fabrication of the strip of the present invention;

FIGS. 8 and 9 are high level flow charts showing a first embodiment of the process of the present invention;

FIG. 10 is a top plan view showing an alternative shape of the single strip of the present invention having patches with planar surfaces;

FIG. 11 is a top plan view of another embodiment of the invention wherein the ultraviolet sensitive patches have been replaced with medicated patches made from a non-woven fabric having blend of salicylic acid and adhesive coated on one side to allow the medicated patches to adhere to the person's skin while applying medication to the affected area of the skin;

FIG. 12 is a cross section view of the medicated patches taken along line 12-12 of FIG. 11;

FIG. 13 is a top plan view of a single strip having a plurality of medicated patches and shown cut from the continuous composite sheet; and

FIG. 14 is a top plan view of a pattern for a cutting die used to perform the full-cut and kiss-cut operations.

### DETAILED DESCRIPTION OF THE INVENTION

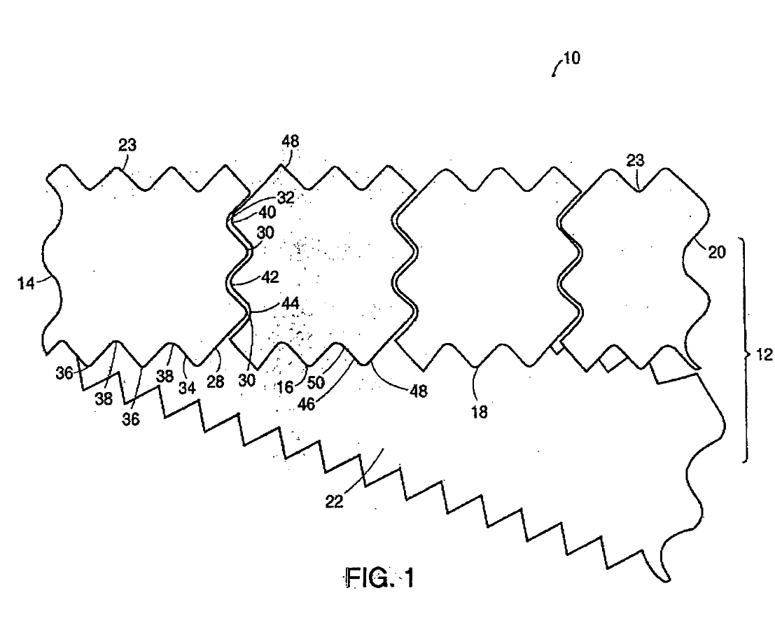

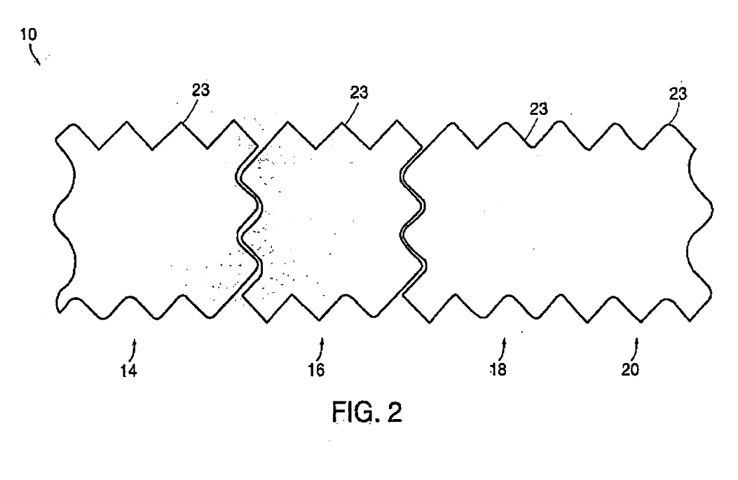

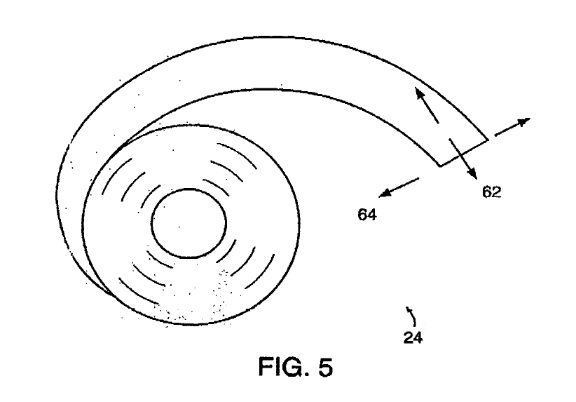

Referring to FIGS. 1-3, an article of manufacture 10 of the present invention is shown for use in alert the person of exposure to an excessive amount of ultraviolet radiation. In one embodiment, the article 10 comprises a strip 12 having a patch 14, a patch 16, a patch 18, and a fourth patch 20, releaseably attached or disposed upon an elongated backing member 22. Multiple strips 12 are manufactured from a continuous roll of material 24 (FIG. 5) fed through a cutting die 26 (to be described) of a feeding machine (not shown). The feeding machine is a web bandage envelope machine available from a variety of suppliers.

Patches 14, 16, 18 and 20 each comprise an ultraviolet sensitive material 23 releaseably connected with the backing member 22. The ultraviolet sensitive material 23 being adapted to change from a first color to a second color indicative of the maximum amount of exposure to ultraviolet radiation. Patches 14, 16, 18 and 20 are of similar design. Patch 14 comprises a first non-planar edge 28 having peaks 30 and valleys 32 and a second non-planar edge 34 having peaks 36 and valleys 38. Patch 14 comprises a third non-planar edge 40 having peaks 42 and valleys 44 and a fourth non-planar edge 46 having peaks 48 and valleys 50. Patches 14 and 16 are nested and adjoined along the first and third non-planar edge 28 and 34, respectively, resulting in substantially zero waste.

In operation, the patches 14 and 16 may be removed as needed from the backing member 22 and adhesively attached to the skin of a person to thereby alert the person that the person has been exposed to said maximum amount of ultraviolet radiation upon the patches 14 and 16 changing from a first color to a second color.

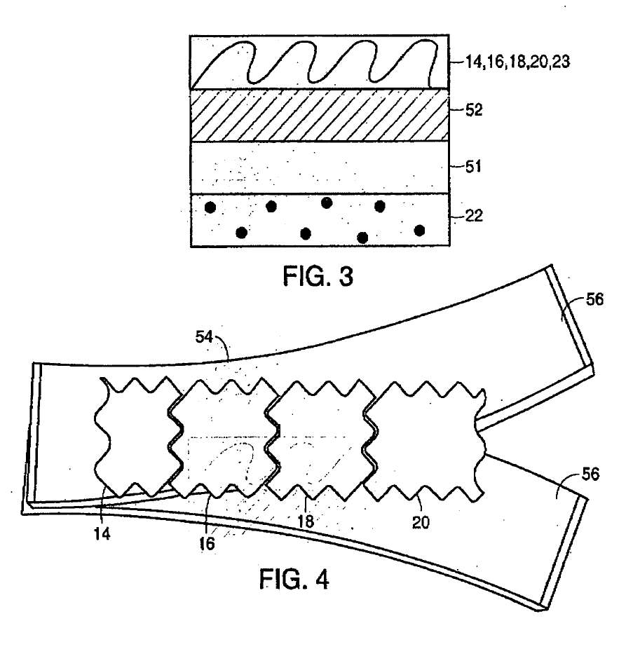

As shown by FIG. 3, the continuous roll comprises the backing member 20, a release material or layer 51, an adhesive coating 52, and the UV sensitive material 22. The release layer 20 is preferably made from a well known Silicon based material. The adhesive coating 22 is preferably made from a human-safe pressure sensitive adhesive widely available from suppliers The UV sensitive material 24 is preferably made from a paper based material treated with an UV sensitive coating widely available from suppliers.

Referring to FIG. 4, wherein the article of manufacture 10 further comprises a sealed enclosure 54 adapted to contain the strip 10. The sealed enclosure 54 is of a design similar to that of enclosures used in connection with conventional bandages and includes a tab 56 enabling a person to open the sealed enclosure 54.

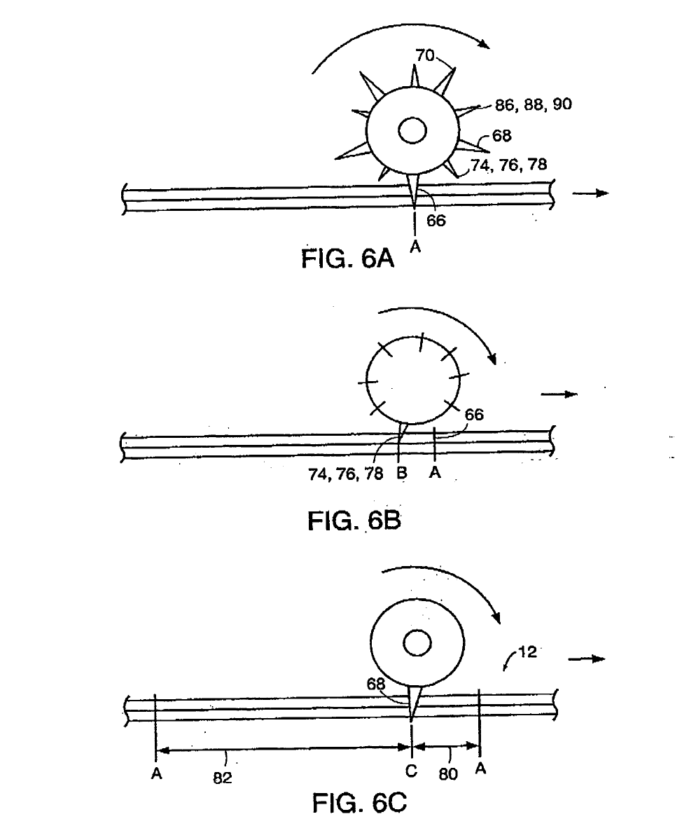

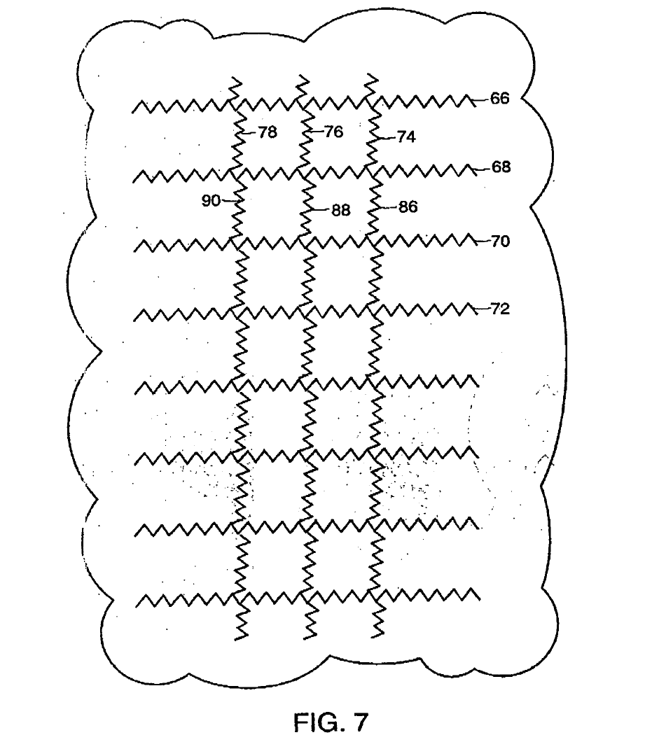

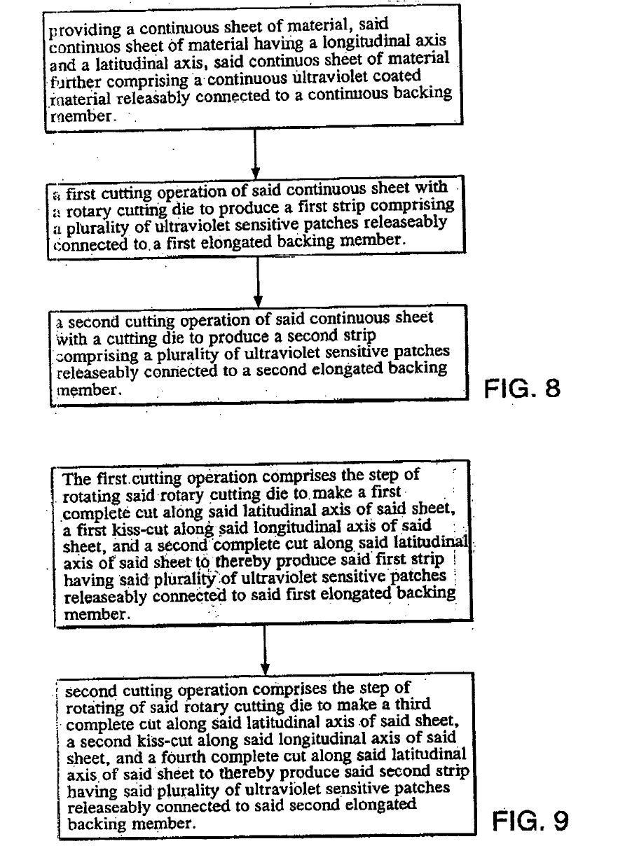

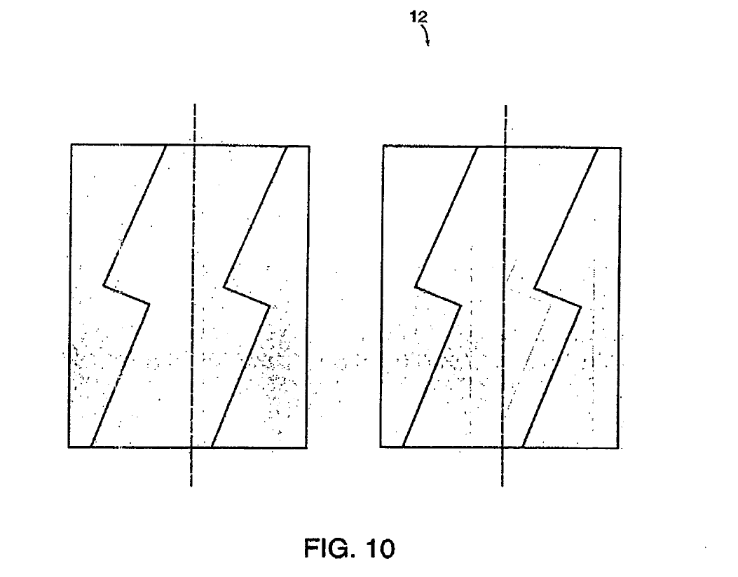

Referring to FIGS. 5, 6A-6C and 7, wherein the cutting die 26 comprises a cutting pattern 60 used in connection with fabrication of the article 10. Cutting die 26 is a rotary cutting die. The roll 24 is oriented such that is controllable fed through the rotary cutting die 26 of the feeding machine (not shown). The roll 24 has a width that is preferably 2.85 inches and is defined by a longitudinal axis 62 and a latitudinal axis 64.

The rotary cutting die 26 comprises complete cut cutting edge 66, 68, 70, and 72, each of which extend a distance equivalent to the width of the roll 24. Th rotary die 26 further comprises a kiss-cut cutting edges 74, 76 and 78.

Upon the feeding of the roll 24 through the rotating rotary cutting die 26 an initial complete cut by the complete cutting edges 66 along the longitudinal axis 64 of the roll 24 occurs which penetrates through the UV coating material 23 and the backing member 22. Upon further rotation of the rotary cutting die 26, a kiss-cut by the kiss-cut cutting edges 74, 76, and 78 occurs which penetrates through the UV coating material 23 but not through the backing material 222 to form a patches 14, 16, 18, and 20 releaseably disposed on the first strip 12. Upon further rotation of the rotary cutting die 26, a complete cut by complete cutting edges 68 is made which penetrates through the UV coating material 23 and the backing material 22 to release the first strip 12 and form one-half of a second strip 84. Upon further rotation of the rotary cutting die 26, a kiss-cut by the kiss-cut cutting edges 86, 88, and 90 which penetrate through the UV coating material 23 but not through the backing material 22 to form patches 14, 16, 18, and 20 releaseably disposed on the second strip 84. Upon further rotation of the rotary cutting die 26, a complete cut by complete cutting edges 70 is made which penetrates through the UV coating material 23 and the backing material 22 to release the second strip 84 and form one-half of the strip 84.

This distance between complete cutting edges 66 and 68 range from 0.7383 inches to 0.7583 inches, and preferably 0.7483 inches in length. This distance between complete kiss-cut cutting edges 74 and 76 and 76 and 78 range from 0.7383 inches to 0.7583 inches, and preferably 0.7483 inches in length having separated patches 14, 16, 18, and 20 that are 0.6400 inches to 0.6600 inches, and preferably 0.6500 inches in width.

Rotation of the rotary cutting die 26 through the cutting operations of the first complete cut, followed by a kiss-cut, and then a second complete cut, results in the creation of the strip 12, wherein the first edge 28 of patch 14 has the peaks 30 and the valleys 32, and wherein the third edge 40 of patch 16 has the peaks 36 and the valleys 38, wherein the second edge 30 of patch 14 has peaks 42 and valleys 44, and the fourth edge 42 of patch 16 having peaks 48 and valleys 50. The distance from peaks 42 and 44 is in the range of 0.1525 inches to 0.1725 inches, preferably 0.1625 inches, and a peak 48 to peak 50 is from 0.1150 inches to 0.1350 inches, preferably 0.1250 inches.

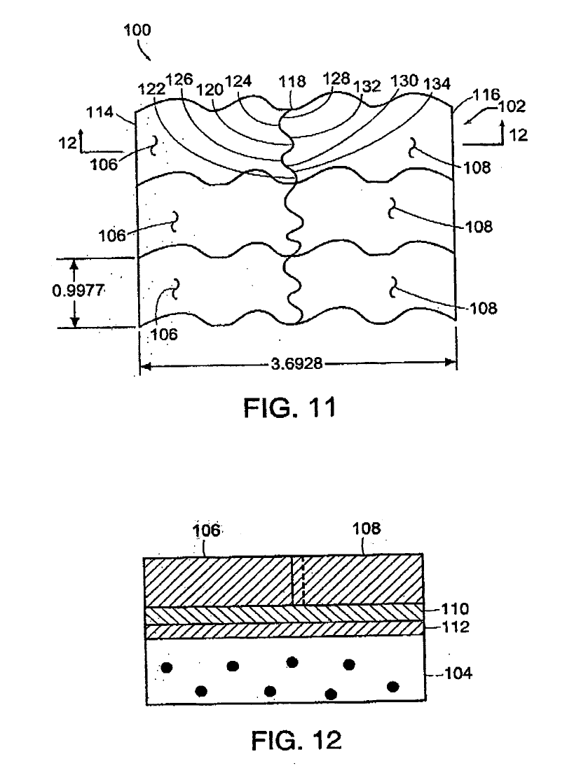

Referring to FIGS. 11-14, wherein another embodiment of the present invention, an article of manufacture 100 is shown for use in removing corns and callus from the skin of a person (not shown). The article 10 generally comprises a strip 102 comprising a backing member 104 having a release material 112 and first and second medicated patches 106 and 108 made from a non-woven fabric having a blend of salicylic acid (40%) and an adhesive coating 110 releaseably affixed to the backing member 104. The backing member 104 and release material 112 may be made from Mylar®. The strip 102 is available as a fully processed and continuous roll of composite sheet material as Part No. 4671 from Akron Coating and Adhesive, Inc, 367 Standon Avenue, Akron Ohio 44301. Each of the first and second medicated patches 106 and 108 comprise first and second outside edges 114 and 116, and an inside edge 118. The first and second medicated patches 106 and 108 are adapted to nest and adjoin along the inside edges 118 of the first and second medicated patches 106 and 108, respectively. The first and second inside edges 118 are non-planar. The inside edge 118 of the first medicated patch 106 comprises first and second peaks 120 and 122, and first and second valleys 124 and 126. The inside edge of the second medicated patch 108 comprises first and second peaks 128 and 130, and first and second valleys 132 and 134. The first and second peaks 120 and 122 of the inside edge 118 of the first medicated patch 106 are nested and adjoined within the first and second valleys 132 and 134 of the inside edge 118 of the second medicated second patch 108.

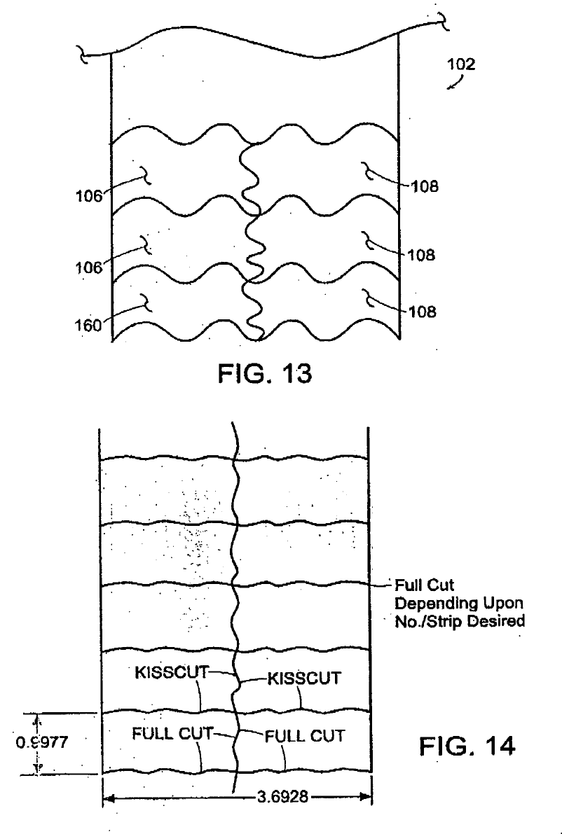

FIG. 13 shows the single strip 102 having three (3) medicated patches 106 and three (3) medicated patches 108 shown cut apart from the strip 102. FIG. 14 shows a pattern for a cutting die used to performing the full-cut and kiss-cut operations as heretofore described in connection with prior embodiments of the present invention.

The foregoing description is intended primarily for purposes of illustration. This invention may be embodied in other forms or carried out in other ways without departing from the spirit or scope of the invention. Modifications and variations still falling within the spirit or the scope of the invention will be readily apparent to those of skill in the art.

What is claimed is:

1. An article of manufacture comprising: a first strip comprising a backing member and first and second medicated patches releaseably affixed to said backing member; each of said first and second patches comprising first and second outside edges and an inside edge; said first and second patches being nested and adjoined along said inside edges of said first and second patches, respectively.

2. The article of manufacture of claim 1, wherein said first and second edges are non-planar.

3. The article of manufacture of claim 2, wherein said inside edge of said first medicated patch comprises first and second peaks and first and second valleys.

4. The article of manufacture of claim 3, wherein said inside edge of said second medicated patch comprises first and second peaks and first and second valleys.

5. The article of manufacture of claim 4, wherein said first and second peaks of said inside edge of said first patch are nested and adjoined within said first and second valleys of said inside edge of said medicated second patch.

6. The article of manufacture of claim 5, wherein said backing member comprises a lower surface having a release material disposed thereon.

7. The article of manufacture of claim 6, wherein said first and second medical patches each comprise an adhesive material disposed upon said lower surface of said first and second medicated patches, respectively.

8. The article of manufacture of claim 7, wherein said first and second medicated patches comprise a solution of salicylic acid.

9. The article of manufacture of claim 8, wherein said first and second medicated patches comprise a solution of beta-hydroxy acid.

10. A process for manufacturing an article of manufacture generally comprising the steps of:
(a) providing a continuous sheet of backing material having a longitudinal axis and a latitudinal axis;
(b) providing a continuous sheet of medicated material having a solution of salicylic acid releaseably affixed to said continuous sheet of backing material;
©) a first cutting operation to cut the sheet using a rotary cutting die to produce a first strip comprising a first plurality of medical patches having releaseably connected to a first backing member; and
(d) a second cutting operation to cut the sheet using the rotary cutting die to produce a second strip comprising a second plurality of medical patches releaseably connected to a second backing member.

11. The process of claim 10, wherein said first cutting operation comprises the step of using the rotary cutting die to make a first complete cut along said latitudinal axis of the sheet, a first kiss-cut along the longitudinal axis of the sheet, and a second complete cut along the latitudinal axis of the sheet to thereby produce the first strip having the first plurality of medicated patches releaseably connected to the first backing member.

12. The process of claim 11, wherein said second cutting operation comprises the step of using the rotary cutting die to make a first complete cut along said latitudinal axis of the sheet, a first kiss-cut along the longitudinal axis of the sheet, and a second complete cut along the latitudinal axis of the sheet to thereby produce a second first strip having the second plurality of medicated patches releaseably connected to the second backing member.
